(19) United States
(12) Patent Application Publication
(10) Pub. No.: US 2016/0159762 A1
(43) Pub. Date: Jun. 9, 2016

(54) PROCESS FOR PRODUCING FURAN FROM FURFURAL FROM BIOMASS

(71) Applicant: ARCHER DANIELS MIDLAND COMPANY, Decatur,, IL (US)

(72) Inventors: Thomas BINDER, Decatur, IL (US); Zheng WANG, Forsyth, IL (US)

(73) Assignee: ARCHER DANIELS MIDLAND COMPANY, Decatur, IL (US)

(21) Appl. No.: 14/906,059

(22) PCT Filed: Jul. 30, 2014
(86) PCT No.: PCT/US14/48783

§ 371 Date: Jan. 19, 2016

Related U.S. Application Data
(60) Provisional application No. 61/864,228, filed on Aug. 9, 2013.

(51) Int. Cl. C07D 307/08; B01J 23/44

(52) U.S. Cl. CPC C07D 307/08; B01J 23/44

(57) ABSTRACT

A process is described for producing furan from furfural from biomass, wherein furfural in an aqueous mass or stream from the liquefaction of biomass or a biomass fraction including one or more furfural precursors is extracted into an organic solvent which is readily separable from furan by simple distillation at atmospheric pressure, furfural is catalytically decarbonylated to furan in the organic solvent and furan is separated from the organic solvent by simple distillation. The furan from the distillation step may be hydrogenated to provide tetrahydrofuran.

### TECHNICAL FIELD

The present invention relates to the production of furan and other products such as tetrahydrofuran from furfural produced from biomass.

### BACKGROUND OF THE INVENTION

Furfural, also known as furan-2-carbaldehyde, is a valuable intermediate in the production of various commercially valuable materials. For example, furfural can be decarbonylated to produce furan, which in turn can be hydrogenated to produce tetrahydrofuran (THF).

About two hundred thousand tonnes of tetrahydrofuran are produced annually, with the primary uses of THF being as a solvent and as a polymer precursor. Thus, for example, THF can be polymerized by strong acids to give a linear polymer called poly(tetramethylene ether) glycol (PTMEG), CAS Registry Number [25190-06-1], also known as PTMO, polytetramethylene oxide. The primary use of this polymer is to make elastomeric polyurethane fibers like Spandex.

The most widely used industrial process for making THF involves the acid-catalyzed dehydration of 1,4-butanediol, akin to the production of diethyl ether from ethanol. The butanediol is derived from condensation of acetylene with formaldehyde followed by hydrogenation. A second route developed by Du Pont produces THF by oxidizing n-butane to crude maleic anhydride followed by catalytic hydrogenation of the maleic anhydride. A third major industrial route entails hydroformylation of allyl alcohol followed by hydrogenation to the butanediol. All of these commercial routes, however, ultimately depend upon feedstocks that are not renewable, being obtained from fossil fuel resources that have in recent years become increasingly costly.

With regard to the present invention, it has long been known that THF can also be synthesized from renewable resources, by dehydrating pentoses found in or obtained from biomass (particularly the hemicellulosic component or fraction of lignocellulosic biomasses) to furfural, decarbonylating the furfural to furan, and then finally hydrogenating the furan to provide THF.

For example, in relation to the final step, U.S. Pat. No. 2,846,449 to Banford et al. (1958) describes a process for producing tetrahydrofuran from furan obtained by the catalytic decarbonylation of furfural and references an earlier DuPont patent to Whitman (U.S. Pat. No. 2,374,149 (1945)) for teaching a method for the vapor phase decarbonylation of furfural to furan in the presence of steam over a catalyst composed of mixed chromites. A second DuPont patent, U.S. Pat. No. 2,776,981 to Tyran, is similarly directed as the Whitman patent, being concerned with the vapor phase decarbonylation of furfural to furan in the presence of steam and using a pelleted chromite catalyst promoted by the addition thereto of an alkali metal ion such as sodium or potassium.

Nevertheless, a renewable method for producing THF has proven elusive, because an economical, practical method of producing a suitable biomass-derived furfural feed for making the furan to be hydrogenated to tetrahydrofuran according to the Banford et al. process or another method has proven elusive. As summarized very recently in US 2013/0168227 to Fagan et al., producing furfural from solid biomass in high yield has been “difficult”, so that furfural conventionally has been produced utilizing biomass such as corn cob or sugar cane bagasse as a raw material feedstock for obtaining glucose, glucose oligomers, cellulose, xylose, xylose oligomers, arabinose, hemicellulose, and other C5 and C6 sugar monomers, dimers, oligomers, and polymers. The hemicellulose and cellulose are hydrolyzed under acidic conditions to their constituent sugars, such as glucose, xylose, mannose, galactose, rhamnose, and arabinose. In a similar aqueous acidic environment, the C5 sugars are subsequently dehydrated and cyclized to furfural. Under similar conditions, C6 sugars can also be hydrolyzed and converted to a limited extent to furfural. In these solid biomass liquefaction methods, a variety of both liquid and solid acids have been proposed for use. As well, various methods of processing the biomass or parts of the biomass (or the liquefaction products from the acid-catalyzed hydrolysis of the biomass or parts/fractions thereof) have been proposed, but as evidenced by a number of recent companion filings to the Fagan et al. published application, see, for example, US 2013/0172581; US 2013/0172582; US 2013/0172583; US 2013/0172584; US 2013/0172584; US 2013/0172585; US 2013/0109869; US2012/0157697; and US2011/0213112 all by the same assignee, there remains a substantial need for further improvement in methods for producing furfural from biomass that will be conducive to the economical realization of a furan product that can then be hydrogenated to THF.

### SUMMARY OF THE INVENTION

The present invention in one aspect concerns an improved method for producing furan from furfural from biomass, wherein furfural in an aqueous mass or stream from the liquefaction of biomass or a biomass fraction including one or more furfural precursors is extracted into an organic solvent which is readily separated from furan by simple distillation, furfural is catalytically decarbonylated to furan in the organic solvent and furan is separated from the organic solvent by simple distillation. In a further aspect, the furan so produced is hydrogenated to THF.

### DETAILED DESCRIPTION OF THE INVENTION

Furfural can conveniently be produced from biomass. It is for example produced in the liquefaction of lignocellulosic material. After the liquefaction of lignocellulosic material it is desirable to separate furfural from the total aqueous product produced. Separation of the furfural by distillation, however, is problematic as furfural can form azeotropes with the water in the total aqueous product.

Alternative approaches to recovering furfural include liquid-liquid extraction processes. U.S. Pat. No. 4,533,743 describes a process for the production of furfural. It describes that state of the art biomass acid hydrolysis processing techniques can breakdown pentosans, a major constituent of biomass hemicellulose, into pentoses. Hot pentose is subsequently reacted in the presence of a mineral acid catalyst in a plug flow reactor at a temperature in the range from 220° C. to 300° C. to produce furfural. The produced furfural is optionally extracted using an essentially water immiscible furfural solvent which does not form an azeotrope with furfural.

As suitable solvents amongst others higher boiling point aromatics, such as diethylbenzene, dipropylbenzene, dimethylethylbenzene, butylbenzene, tetralin and isophorone; aromatics, such as toluene; halogenated aromatics; and also halogenated alkanes are mentioned.

U.S. Pat. No. 6,441,202 describes a method to produce sugars by acidic hydrolysis of biomass and subsequently subject the sugars to dehydration to form a hydrolysate comprising heterocyclic compounds such as furfural and hydroxymethylfurfural and acid. Subsequently the heterocyclic compounds are extracted from the hydrolysate by a hydrocarbon. The acid may include an organic or inorganic acid, such as for example sulfuric acid and the hydrocarbon may for example be toluene.

FR 2411184 also describes a process for the preparation of furfural. It describes submitting of a sugar solution to acid dehydration to convert xylose into furfural. The furfural is extracted with a solvent. As suitable solvents amongst others toluene, xylene, methyl-naphthalene and benzaldehyde are mentioned.

J. Croker et al. describe a process for liquid extraction of furfural from an aqueous solution (see their article “liquid extraction of furfural from aqueous solution” by John R. Croker and Ron G. Bowrey, Ind. Eng. Chem. Fundam. 25, vol.23, pages 480-484 (1984)). They describe extraction for water-furfural methyl isobutyl ketone; water-furfural-isobutyl acetate and water-furfural-toluene systems.

Whether a biphasic approach is taken in the manner of these references or whether efforts are made to separate furfural from the aqueous liquefaction product directly by distillation or like methods, notwithstanding the tendency of furfural to form an azeotrope with water in the aqueous liquefaction product, to the best of Applicants' knowledge the prior art has not described forbearing any attempt to recover the furfural and instead converting furfural to furan, then recovering the furan product rather than the furfural.

We have found that by employing a liquid-liquid extraction method to remove furfural from an aqueous mass or stream from the liquefaction of biomass or a biomass fraction including one or more furfural precursors (principally meaning those substances found in biomass than can be acidically- or enzymatically hydrolyzed to their constituent C5 sugars but also including substances that will yield C6 sugars) into an organic solvent that is readily separable from furan by simple distillation, and catalytically decarbonylating furfural so extracted to furan while in the organic solvent medium, the furfural value from the biomass can be more simply recovered by a simple distillation to separate the furan from the organic solvent.

A number of organic solvents may be considered among those that have been previously suggested as useful for a biphasic approach to recovering furfural from an aqueous biomass liquefaction product, but we have found toluene works quite satisfactorily, having a boiling point under standard atmospheric conditions that is approximately 80 degrees Celsius greater than furan (110.6 degrees Celsius versus 31.3 degrees Celsius). A number of decarbonylation catalysts have likewise been evaluated and described for the liquid phase decarbonylation of furfural, including various supported and promoted or unpromoted platinum, rhodium, palladium and nickel catalysts, see also for example U.S. Pat. No. 4,780,552, but we have found a supported palladium catalyst of a type widely described in the literature for this purpose works quite satisfactorily. Other preferred aspects of carrying out the decarbonylation are described in the examples that follow, though certainly those skilled in the art will be well able given previous investigations into the liquefaction and processing of biomass to produce furfural, the recovery of furfural from an aqueous mass or stream from the liquefaction and processing of the biomass into an organic solvent, and finally the decarbonylation of furfural in a liquid phase to further refine and optimize the production of furan by means of the inventive process without departing from the scope of the present invention as defined by the claims following hereafter.

Once the furfural is decarbonylated, the furan product is preferably separated from the organic solvent by simple distillation. The furan may then according to the second aspect be hydrogenated to THF, for example, using any of the conventionally known methods for accomplishing the hydrogenation. U.S. Pat. No. 2,846,449 to Banford et al. prescribes finely divided nickel, platinum or palladium in the pure state or on an inert support, with foraminous or Raney nickel and finely divided reduced nickel or kieselguhr being their preferred catalyst choices.

The present invention is further demonstrated by the examples that follow:

### EXAMPLES

Set up: All tests were done in a 300 ml batch reactor. High purity N2 was used for flushing the system.

Feed: Most of the tests were done under synthetic feed made from commercially available furfural and toluene. In each test, we used 7.5 grams furfural and 142.5 grams toluene which gives 5% furfural in toluene. Other tests were done using the toluene phase of the dehydration product.

Catalyst: Two commercially available catalysts were used. One was 1% Pd/Al203 and the other was 2% Pd/C. If not specifically noted, the test was done on the 2% Pd/C catalyst.

Temperature: Most of the decarbonylation tests were done at 250 deg. C. We also tried 200 and 230 deg. C., but 250 deg. C. gave the best yield.

Pressure: Only the initial pressure at room temperature was controlled. We typically controlled it at 30 psi. During testing the system was closed.

Reaction time: Longer reaction times were not helpful absent avoiding high CO concentrations in the gas phase. With proper releasing of CO produced by the decarbonylation of furfural, high yields were observed with a reaction time of about 3 hours.

### Results and Discussion

### Temperature Effect

As shown in Table 1, a higher reaction temperature favored a higher yield on both catalysts tested.

TABLE 1

| Catalyst | Temperature (° C.) | Furan yield |
|---|---|---|
| 1% Pd/Al2O3 | 200 | 36% |
| | 230 | 70% |
| 2% Pd/C | 230 | 63% |
| | 250 | 83% |

### Pressure Effect

The decarbonylation of one mole of furfural produces one mole of furan and one mole of carbon monoxide (CO). The accumulation of CO in the closed system was found to inhibit the reaction from going to higher conversion. One way to lower the CO partial pressure in the system was to lower the initial N2 pressure in the reactor. We found that when the initial nitrogen pressure was reduced from 330 psi to 30 psi, furan yield increased from 70% after 3.5 hours reaction time to 79% after 3 hours reaction time.

### Effect of N2 Purging

To keep the CO concentration in the gas phase low, we also tried purging the system with N2 after a certain time of reaction. However, directly purging the system at the reaction temperature (200-250 C) will take toluene and furan out as well, thus the purging step was done at room temperature.

Typically we instituted a series of room temperature nitrogen purges preceding reaction at temperature for an interval. Without N2 purging, we were able to achieve 79% yield after 3 hours' reaction time. With several iterations of purging with 300 psi nitrogen before reducing the nitrogen pressure to 30 psi for the start of the reaction interval (to ensure nitrogen only filled the headspace and to displace any other dissolved gases in the liquid phase (whether CO, oxygen or other) with nitrogen to the extent possible) and before heating to reaction temperature, the best result with the 2% Pd/C catalyst at 250 deg. C. reaction temperature was 83% yield after a first 80 minutes of reaction time and almost complete conversion of the furfural after a second series of high pressure nitrogen purges and a further reaction time of 60 minutes.

### Catalyst Stability

To check stability of the catalysts, we used the same catalyst in a series of different batch runs and did observe a reduction in yield from earlier to later batches. All reactions were done at 250 deg C. with clean feed. Nothing was done to the filtered and recovered catalysts between batches, and filtration losses between batches were negligible.

The results are shown in Table 2. In the first batch run with fresh catalyst, complete conversion was obtained after two reaction intervals with the intervening nitrogen purging as described above. In the second batch run with once-used catalyst complete conversion was achieved after three intervals, while only 71% yield was achieved in the third batch run with the same catalyst after three reaction intervals.

TABLE 2

| | | Second | |
|---|---|---|---|
| | First batch | Batch | Third batch |
| 1st step | 83% | 63% | 40% |
| 2nd step | 99% | 80% | 53% |
| 3rd step | | 100% | 71% |

### Performance with Actual Dehydration Feed

We were able to achieve complete conversion of furfural in the toluene extract of a dehydrated pentose-containing feed from biomass at 250 deg C. after three reaction intervals with fresh catalyst. We further used the same catalyst to run another batch with clean feed. Results are shown in Table 3, and by comparison with Table 2 using a synthetic furfural feed, lower yields were achieved after the 2nd and 3rd intervals with the actual dehydration feed suggesting some degree of increased deactivation of the catalyst with using the toluene extract from an actual dehydration feed.

TABLE 3

| | First batch with | First batch with |
|---|---|---|
| | Actual Feed | Synthetic Feed |
| 1st step | N/A | 63% |
| 2nd step | 55% | 80% |
| 3rd step | 74% | 100% |

We also ran both of the toluene extract from the actual dehydration product and the synthetic toluene extract using the 1% Pd/Al₂O₃ catalyst. Results are from fresh catalyst on a single batch. As shown in Table 4, lower yield was again seen after each reaction interval with the toluene extract of the actual dehydration product.

TABLE 4

| | Synthetic Feed | Actual feed |
|---|---|---|
| 1st step | 70% | 52% |
| 2nd step | 85% | 68% |
| 3rd step | 93% | 75% |

What is claimed is:

1. A process for producing furan from furfural from biomass, wherein furfural in an aqueous mass or stream from the liquefaction of biomass or a biomass fraction including one or more furfural precursors is extracted into an organic solvent which is readily separable from furan by simple distillation at atmospheric pressure, furfural is catalytically decarbonylated to furan in the organic solvent and furan is separated from the organic solvent by simple distillation.

2. A process according to claim 1, further comprising the step of hydrogenating furan from the simple distillation to produce tetrahydrofuran.

3. A process according to claim 1 or claim 2, wherein toluene is used to extract furfural from the aqueous mass or stream.

4. A process according to claim 1 or claim 2, wherein furfural is decarbonylated to furan in the presence of a supported palladium catalyst.

5. A process according to claim 4, wherein the support is carbon or alumina.

6. A process according to claim 2, wherein furan is hydrogenated to tetrahydrofuran in the presence of a Raney nickel catalyst.
